(19) United States
(12) Patent Application Publication
(10) Pub. No.: US 2019/0082941 A1
(43) Pub. Date: Mar. 21, 2019

(54) CAMERA CONTROL UNIT FOR ENDOSCOPE AND ENDOSCOPE SYSTEM

(71) Applicant: Olympus Corporation, Tokyo (JP)

(72) Inventors: Nanako Ubayama, Tokyo (JP); Takehide Fujimoto, Tokyo (JP); Hitoshi Komine, Tokyo (JP)

(73) Assignee: Olympus Corporation, Tokyo (JP)

(21) Appl. No.: 16/191,637

(22) Filed: Nov. 15, 2018

Related U.S. Application Data
(63) Continuation of application No. PCT/JP2017/012946, filed on Mar. 29, 2017.

(30) Foreign Application Priority Data
May 24, 2016 (JP) 2016-103447

(51) Int. Cl. A61B 1/045; G02B 23/24; A61B 1/00; A61B 1/05; A61B 1/06

(52) U.S. Cl. CPC A61B 1/045; G02B 23/2484; A61B 1/00006; A61B 1/00096; A61B 1/05; A61B 1/0661; A61B 1/00009

(57) ABSTRACT

A camera control unit for an endoscope includes a power supply, an identifying portion, a control portion, and a switch. The power supply generates a required voltage for each of the plurality of various endoscopes expected to be connected to the camera control unit. The identifying portion identifies the required voltage of one of the various endoscope connected to the camera control unit. The control portion controls the power supply in such a manner that power having the required voltage is generated according to an identification result of the identifying portion. The switch is disposed on a power supply path connected to the power supply. The switch is set to alternate between conduction and/or blocking of the power supply path.

### CROSS-REFERENCE TO RELATED APPLICATIONS

This application is a continuation application of PCT Application No. PCT/JP2017/012946 filed on Mar. 29, 2017, which in turn claim priority to the Japanese Patent Application No. 2016-103447 filed on May 24, 2016 in Japan which is hereby incorporated by reference in its entirety.

### TECHNICAL FIELD

The technology disclosed herein relates to a camera control unit for an endoscope and an endoscope system that provides multiple power supplies to an endoscope.

### DESCRIPTION OF THE RELATED ART

In recent years, endoscope apparatus has been used in various fields such as medical field and industrial field for example. In the medical field, the endoscope apparatus is used, for example, for observation of an organ in a body lumen, treatment procedure with use of a procedure instrument, surgery under endoscope observation, and so forth. As the endoscope apparatus, an electronic endoscope is configured to be capable of imaging of a captured image in a patient body lumen by an imaging element. The endoscope apparatus has a camera control unit that executes video processing of the captured image obtained by imaging component of the electronic endoscope. The camera control unit then converts the captured image to a video signal and output the video signal to a monitor and record the video signal.

The endoscope is attachably/detachably connected to the camera control unit via a cable. The imaging element disposed in the endoscope supplies a captured image to the camera control unit via the cable. The imaging element receives power supply from the camera control unit. The endoscope is equipped with components necessary for power supply besides the imaging element. The power is provided from the camera control unit to the respective components of the endoscope via multiple power supply lines.

Incidentally, the endoscope is equipped with components different for each kind of endoscope. For example, the kind of imaging element often differs for each kind of endoscope. The voltage necessary for driving is not common to such different kinds of components in some cases. In the cases, various kinds of endoscopes are connected to a camera control unit. For this reason, the configuration needs to be made in such a manner that different supply voltages according to the respective components can be provided via one power supply line, so that the number of mounted power supplies increases.

Therefore, in JP 2010-088656A, an apparatus is disclosed. The number of power supplies is set smaller in the apparatus by employing power supplies that adjust supply voltage according to the endoscope type and carry out power supply to endoscopes.

However, in the apparatus of JP 2010-088656A, there is a problem that a predetermined period is required for adjustment of the output voltage from the power supply. Also, the supply voltage in this adjustment period often adversely affects a component of the endoscope.

The present disclosure intends to provide a camera control unit for an endoscope and an endoscope system that can prevent components of an endoscope from being adversely affected even in the case of employing power supplies that can adjust the output power of the power supplies and provide multiple output voltages to the endoscope.

### BRIEF SUMMARY OF EMBODIMENTS

A camera control unit for an endoscope according to one aspect of the present disclosure is a camera control unit for an endoscope to which a plurality of various endoscopes different voltage requirement is connectable. The camera control unit includes a power supply, an identifying portion, a control portion, and a switch. The power supply generates a required voltage for each of the plurality of various endoscopes expected to be connected to the camera control unit. The identifying portion identifies the required voltage for one of the plurality of the various endoscopes connected to the camera control unit. The identifying portion outputs an identification result. The control portion controls the power supply in such a manner that power having the required voltage is generated according to the identification result. The switch is disposed on a power supply path connected to the power supply. The switch is set to alternate between conduction and/or blocking of the power supply path.

Furthermore, an endoscope system according to one aspect of the present disclosure is an endoscope system having an endoscope and a camera control unit. A plurality of various endoscopes each of which having different voltage requirement are connectable to the camera control unit. The endoscope system having the camera control unit as mentioned hereinbefore also includes a power supply, an identifying portion, a power supply control portion, and a switch. The power supply generates a required voltage of each for the plurality of the various endoscopes expected to be connected to the camera control unit. The identifying portion identifies the required voltage for one of the plurality of the various endoscopes connected to the camera control unit. The identifying portion outputs an identification result. The control portion controls the power supply in such a manner that power having the required voltage is generated according to the identification result. The switch is disposed on a power supply path connected to the power supply. The switch is set to alternate between conduction and/or blocking of the power supply path.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a block diagram depicting a camera control unit for an endoscope according to a first embodiment of the technology disclosed herein.

FIG. 2 is a perspective view depicting one example of the configuration of an endoscope.

FIG. 3 is a flowchart for explaining operation of the first embodiment.

FIG. 4 is a timing chart for explaining the operation of the first embodiment.

FIG. 5 is a block diagram depicting a second embodiment of the technology disclosed herein.

FIG. 6 is a flowchart for explaining operation of the second embodiment.

FIG. 7 is a block diagram depicting a third embodiment of the technology disclosed herein.

FIG. 8 is a flowchart for explaining operation of the third embodiment.

FIG. 9 is a block diagram depicting a fourth embodiment of the technology disclosed herein.

FIG. 10 is a block diagram depicting a fifth embodiment of the technology disclosed herein.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

Embodiments of the technology disclosed herein will be described in detail hereinafter with reference to the drawings.

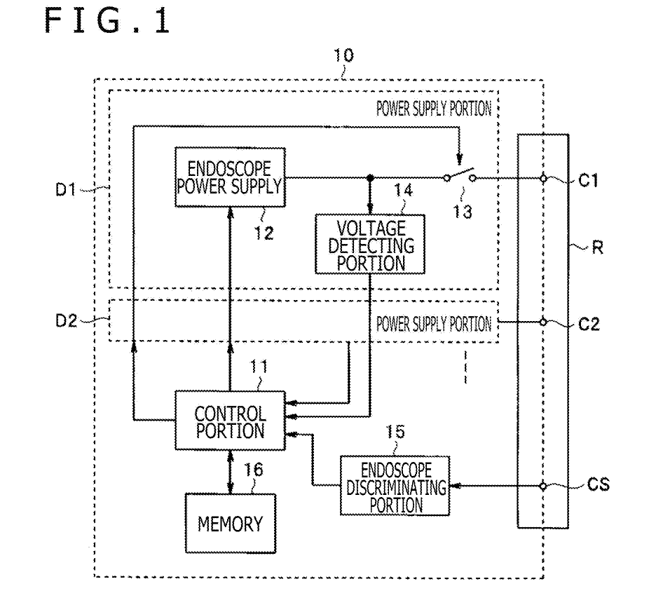

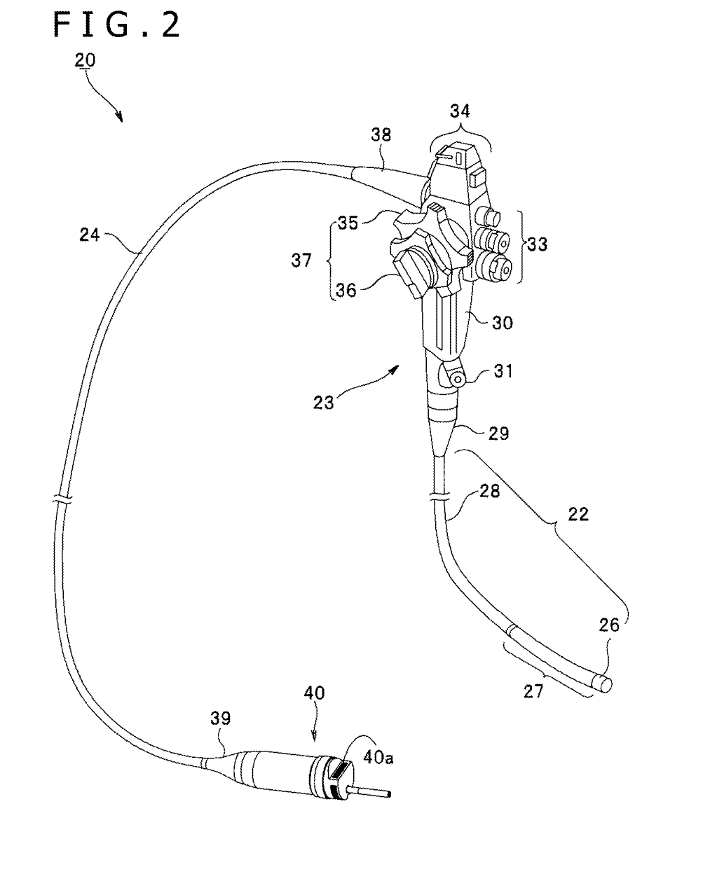

FIG. 1 is a block diagram depicting a camera control unit for an endoscope according to a first embodiment of the present invention. Furthermore, FIG. 2 is a perspective view depicting one example of the configuration of an endoscope.

As depicted in FIG. 2, an endoscope 20 has an insertion portion 22, an operation portion 23, a universal cable 24, and an endoscope connector 40. The insertion portion 22 is an elongated long member inserted into an observation-target region, e.g. a lumen such as a large intestine. The operation portion 23 is disposed continuously with the proximal portion of this insertion portion 22. The universal cable 24 is a composite cable extended from a side surface of this operation portion 23. The endoscope connector 40 is a connector that is disposed at an end portion of the universal cable 24. The endoscope connector 40 is attachably/detachably connected to light source apparatus and a camera control unit (hereinafter, referred to as CCU) 10. The endoscope 20 also includes a plurality of circuit components that operate by a predetermined required voltage.

The insertion portion 22 of the endoscope 20 has a distal portion 26. An imaging portion using a charge coupled device (CCD) or complementary metal oxide semiconductor (CMOS) sensor or the like is incorporated on the distal side of the distal portion 26. A bending portion 27 is a movable portion that can freely bend and is disposed continuously with the rear portion of this distal portion 26. Moreover, a flexible tube portion 28 that is formed of a soft tubular member and is long and has flexibility is disposed continuously with the rear portion of this bending portion 27. The proximal portion of the flexible tube portion 28 of this insertion portion 22 is connected to a breaking prevention portion 29 of the operation portion 23.

The operation portion 23 includes a grip portion 30 gripped by a user at the time of use. A procedure instrument insertion port 31 forms a proximal opening of a procedure instrument channel. The procedure instrument channel is disposed in the insertion portion 22. The breaking prevention portion 29 and the grip portion 30 are continuously disposed across the part. The procedure instrument insertion port 31 is disposed at the part. Furthermore, a bending operation portion 37 is disposed at the grip portion 30 of the operation portion 23. The bending operation portion 37 has two, here, bending operation knobs 35 and a fixing lever 36. Bending operation of the bending portion 27 of the insertion portion 22 is carried out with the bending operation knobs 35. The fixing lever 36 fixes these bending operation knobs 35 at a desired rotational position. Moreover, switches 33 and 34 for operating various kinds of endoscope functions are disposed at the grip portion 30.

The universal cable 24 has breaking prevention portions 38 and 39 at both end portions connected to the operation portion 23 or the connector 40. The breaking prevention portions 38 and 39 keeps the connection strength in such a manner as to cover the outer circumferential portion of the universal cable 24 to prevent damage due to a twist or the like. The connector 40 is attached to the breaking prevention portion 39 of the terminal of the universal cable 24.

The connector 40 is connected to a receptacle portion R of the CCU 10 of FIG. 1 and thereby the endoscope 20 and the CCU 10 are electrically connected. Various kinds of endoscopes other than the endoscope 20 of FIG. 2 can be connected to the CCU 10. However, the endoscope 20 refers to various kinds of endoscopes that can be connected to the CCU 10 hereinafter. In the endoscope 20, one or more components that each operate by a predetermined supply voltage, e.g. field-programmable gate array (FPGA), imaging element, and so forth, are incorporated.

In FIG. 1, only a power supply system of the CCU 10 is depicted and diagrammatic representation is omitted regarding the other part such as an image processing portion. The receptacle portion R of the CCU 10 has an insertion portion with an inner shape corresponding to the outer shape of a plug portion at the tip of the connector 40 (diagrammatic representation is omitted. Multiple terminals 40a are connected to the respective terminals of the receptacle portion R through insertion of the connector 40 into the receptacle portion R. Multiple power supply terminals, terminals for type determination, terminals for connection detection, and so forth are disposed at the connector 40 and the receptacle portion R and are corresponding in each other. When the connector 40 is inserted into the receptacle portion R, the corresponding terminals are electrically connected to each other. In FIG. 1, only power supply terminals C1 and C2 and a terminal CS for type determination among multiple terminals disposed at the receptacle portion R are depicted.

As described hereinbefore, various kinds of electronic components are mounted in the endoscope 20. The various kinds of electronic components need power supply and are not depicted in the diagram. These electronic components are each supplied with power from a respective one of power supply lines. These multiple power supply lines forms a power supply path. The power supply lines are inserted in the endoscope 20 and are not depicted in the diagram. The power supply terminals C1, C2, . . . (hereinafter, referred to as the power supply terminal C if they do not need to be discriminated) are each connected to a respective one of the power supply lines disposed in the endoscope 20 via the connector 40. The number of terminals of the power supply terminals C1, C2, . . . of the CCU 10 is set to a number corresponding to the number of power supplies needed in the connectable endoscope (maximum number).

A control portion 11 is disposed in the CCU 10. The control portion 11 controls the respective portions of the CCU 10. For example, the control portion 11 may be formed of an FPGA or may be formed of a processor such as a central processing unit (CPU).

In the CCU 10, power supply portions D1, D2, . . . the number of which corresponds to the number of power supplies needed in the endoscope are disposed. The power supply portions D1, D2, . . . have the same configuration as each other and the output terminals of the power supply portions D1, D2, . . . are connected to the power supply terminals C1, C2, . . . , respectively, of the receptacle portion R. Hereinafter, if the power supply portions D1, D2, . . . do not need to be discriminated, they will be described as the power supply portion D as a representative. The power supplies, the power supply paths, and the switches the numbers of which correspond to the number of the circuit components of the endoscope 20 are disposed.

An endoscope power supply 12 is disposed in the power supply portion D. The endoscope power supplies 12 generate supply voltages of the respective required voltage values used in the endoscope 20. The endoscope power supply 12 is a variable power supply. The endoscope power supply 12 is controlled by the control portion 11 as a power supply control portion and the generated supply voltage is adjusted. The “power supply output” of the endoscope power supply 12 is provided to the endoscope 20 via the power supply terminal C.

In the present embodiment, a switch 13 is disposed in each power supply portion D. The switch 13 blocks the provision of the “power supply output” from the endoscope power supply 12 to the power supply terminal C. For example, the switch 13 is disposed on a power supply line between the output terminal of the endoscope power supply 12 and the power supply terminal C. The switch 13 can permit or block the provision of the “power supply output” from the endoscope power supply 12 to the endoscope 20 by being on- or off-controlled by the control portion 11 as a switch control portion and electrically connecting or disconnecting the power supply line.

In the present embodiment, a voltage detecting portion 14 is disposed in each power supply portion D. The voltage detecting portion 14 detects the voltage of the “power supply output” from the endoscope power supply 12. The detecting portion 14 outputs the detected result to the control portion 11.

Furthermore, an endoscope identifying portion 15 is disposed in the CCU 10. The endoscope 20 holds information that represents the type of the endoscope (hereinafter, referred to as type information). The endoscope 20 can output the type information to the CCU 10 through a terminal for “type determination” by a resistor voltage division system, a communication system, or the like. The terminal is disposed at the connector 40. The type information from the endoscope 20 is given to the endoscope identifying portion 15 via the terminal CS for type determination. The endoscope identifying portion 15 determines the type of the connected endoscope based on the type information and outputs the determination result to the control portion 11. When the connector 40 is connected to the receptacle portion R, a voltage appears in the terminal for connection detection, which is not depicted in the diagram, and is supplied to the control portion 11. The control portion 11 can detect that the endoscope 20 is connected to the CCU 10.

A memory 16 is storing portion. In the memory 16, information on the supply voltage is stored for each power supply portion D. The supply voltage should be generated corresponding to the type of the connected endoscope. The control portion 11 reads out the information on the supply voltage that should be generated in the endoscope power supply 12 (hereinafter, referred to as target output voltage or required voltage) from the memory 16. The control portion 11 controls the endoscope power supply 12 based on the determination result of the endoscope type. When the target output voltage is specified by the control portion 11, the endoscope power supply 12 adjusts the output power to generate the specified target output voltage.

The control portion 11 determines whether or not the “power supply output” with the specified target output voltage is generated from the endoscope power supply 12 based on the detection result of the voltage detecting portion 14. The control portion 11 determines whether or not the present period is an “adjustment period” based on the detection result of the voltage detecting portion 14. The output voltage is adjusted in the endoscope power supply 12 in the adjustment period in order to generate the specified target output voltage. The control portion 11 sets the switch 13 to the off-state in the adjustment period. After the end of the adjustment period, i.e. after the target output voltage has come to be generated from the endoscope power supply 12, the control portion 11 turns on the switch 13 to provide the power supply output from the endoscope power supply 12 to the endoscope 20.

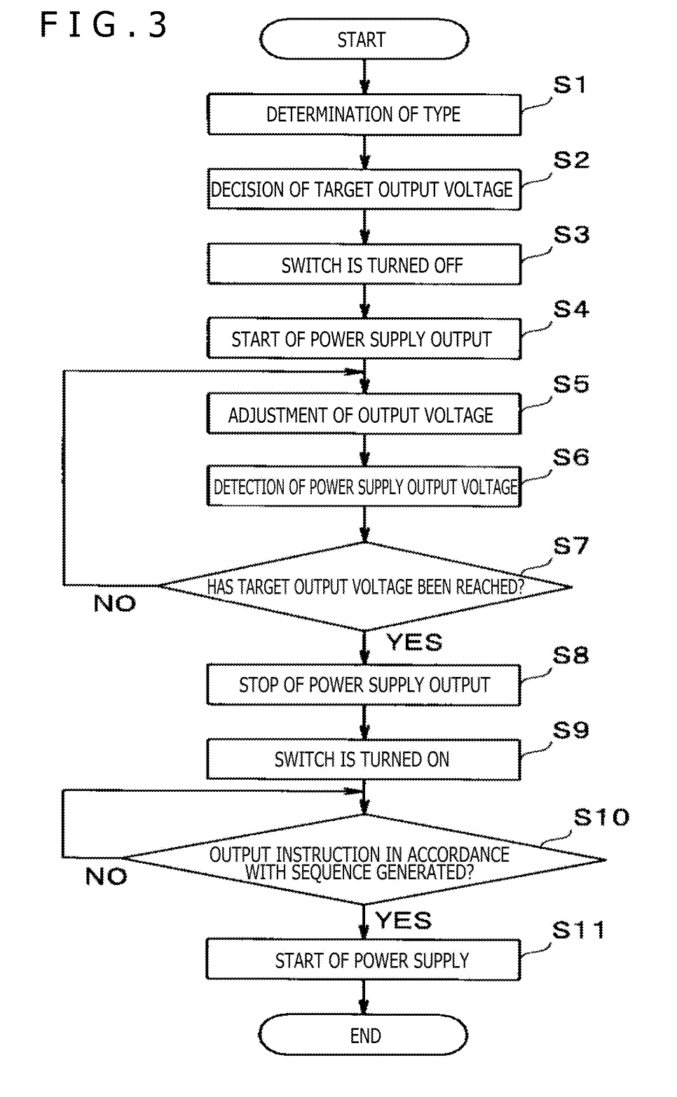

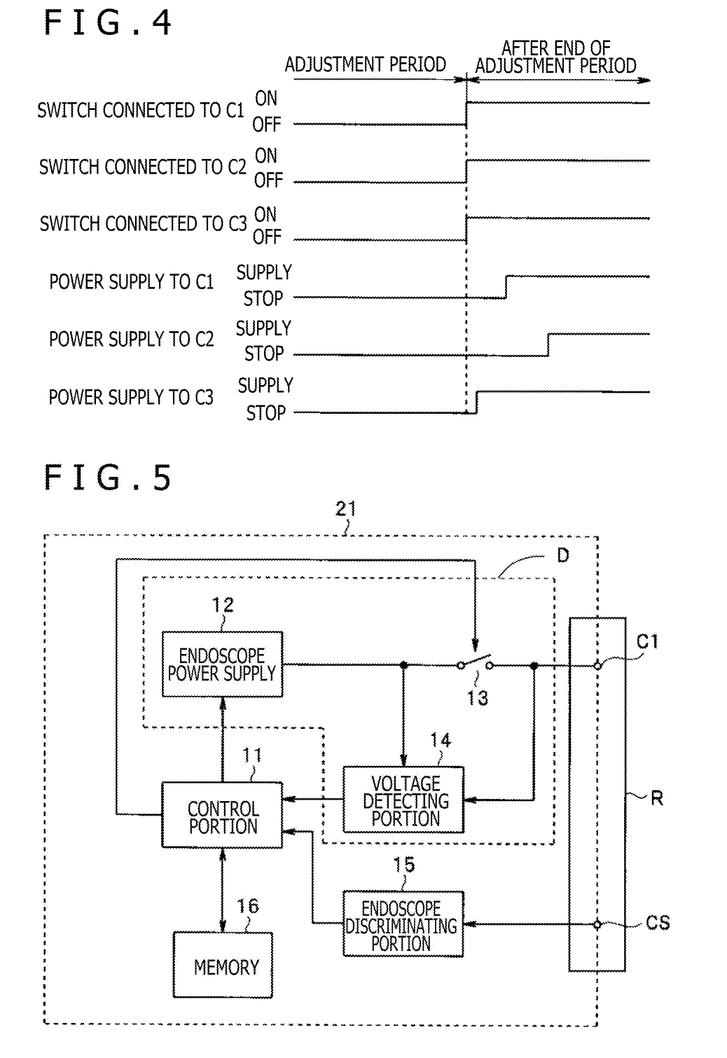

Next, operation of the embodiment configured in this manner will be described with reference to FIG. 3 and FIG. 4. FIG. 3 is a flowchart for explaining operation of the first embodiment and FIG. 4 is a timing chart for explaining the operation of the first embodiment.

The CCU 10 is powered on and thereby the respective portions of the CCU 10 start operation. The control portion 11 determines whether or not the connector 40 of the endoscope 20 has been connected to the receptacle portion R by monitoring the voltage that appears in the terminal for connection detection. When the connector 40 is inserted into the receptacle portion R, the control portion 11 controls the endoscope identifying portion 15 to cause the endoscope identifying portion 15 to determine the type of the connected endoscope in Step S1 in FIG. 3. The endoscope identifying portion 15 determines the type of the connected endoscope 20 based on the type information input via the terminal CS for type determination. The endoscope identifying portion 15 outputs the determination result to the control portion 11.

In Step S2, the control portion 11 reads out the target output voltage value according to the type of the connected endoscope from the memory 16 regarding each power supply portion D. Next, the control portion 11 turns off the switch 13 of each power supply portion D in Step 3. Thereafter, the control portion 11 sets each of the read-out target output voltage values in the endoscope power supply 12 of a respective one of the power supply portions D. The control portion 11 causes each endoscope power supply 12 to start generation of the power supply output in Step 4.

The endoscope power supplies 12 adjust the output voltages so that the target output voltages may be obtained in Step 5. Also in this adjustment period, the power supply outputs are output from the output terminals of the endoscope power supplies 12. However, in this adjustment period, the switches 13 are in the off-state. The power supply outputs from the endoscope power supplies 12 are not provided to the endoscope 20.

In Step S6, in each power supply portion D, the voltage detecting portion 14 detects the supply voltage generated at the output of the endoscope power supply 12. The voltage detecting portion 14 outputs the detection result to the control portion 11. The control portion 11 determines whether or not each power supply output has reached a respective one of the target output voltages in Step S7. When the power supply outputs of the output terminals of the endoscope power supplies 12 have reached the target output voltages, the control portion 11 makes a transition of the processing to Step S8 to temporarily stop the power supply outputs of the endoscope power supplies 12. Next, the control portion 11 turns on the switches 13 in Step S9.

In FIG. 3, control to temporarily stop the power supply outputs of the endoscope power supplies 12 is carried out before the switches 13 are turned on. However, the switches 13 may be turned on without temporarily stopping the power supply outputs of the endoscope power supplies 12. For example, the switches 13 of the respective power supply portions D may be sequentially turned on in accordance with a predetermined sequence. However, when the switch 13 is turned on in the state in which the power supply output is being generated, there is a possibility that (i) a comparatively-large “inrush current” is supplied to the endoscope 20 or (ii) distortion is caused in the supply voltage waveform due to the influence of the “inrush current”. Normally, the endoscope power supply 12 has a so-called soft start function to cause the power supply output to gradually rise up. It is possible to more suppress trouble due to the inrush current when this soft start function is used and the power supply output is generated from the endoscope power supply, with the switch 13 being in the on-state. For this reason, in the present embodiment, the power supply outputs of the endoscope power supplies 12 are temporarily stopped before the switches 13 are turned on.

For example, in the adjustment period, the control portion 11 (i) generates an “adjustment value” for causing the power supply output of the endoscope power supply 12 so as to gradually rise up and (ii) stores the adjustment value in the memory 16. The control portion 11 controls to turn on the switch 13 after the adjustment value is stored in the memory 16. At the time of the start of generation of the power supply output from the endoscope power supply 12, the control portion 11 enables soft start by controlling the endoscope power supply 12 based on the adjustment value read out from the memory 16. In other words, the control portion 11 generates the power having the required voltage according to the identification result based on the “adjustment value” stored in the memory 16 after the switch 13 is turned on.

As described hereinbefore, the CCU 10 has the multiple power supply portions D corresponding to the number of power supplies necessary for the endoscope 20. The control portion 11 controls the endoscope power supplies 12 of the respective power supply portions D so as to generate the respective power supply outputs necessary for the endoscope 20 in a predetermined sequence.

The control portion 11 determines whether or not the timing of generation of an output instruction in accordance with the predetermined sequence has been reached regarding each power supply portion D in Step S10. If the timing of generation of an output instruction to the endoscope power supply 12 has been reached, the control portion 11 instructs the endoscope power supply 12 so as to generate the power supply output. In this manner, the power supply output generated by the endoscope power supply 12 is provided to the endoscope 20 via the switch 13 and the corresponding power supply terminal C. The power supply output is provided to an electronic component such as the imaging element via the corresponding power supply line in the endoscope 20 in Step S11.

FIG. 4 depicts one example of this sequence control. FIG. 4 depicts an example in which three power supply portions D1 to D3 are disposed in the CCU 10. In the example of FIG. 4, the switches 13 are connected to the power supply terminals C1 to C3 and are in the off-state in the adjustment period and are simultaneously turned on after the end of the adjustment period. In FIG. 4, the end of the adjustment period of the power supply portions D1 to D3 is made to correspond with the end of the adjustment period of the power supply portion D in which the adjustment period ends the latest. The switches 13 are simultaneously turned on. The power supply portions D1 to D3 may be allowed to each independently turn on the switch 13 due to the end of the adjustment period. As just described, power supply from the CCU 10 to the endoscope 20 is blocked by the switches 13 in the adjustment period.

Each endoscope power supply 12 of the power supply portions D1 to D3 generates the power supply output based on a signal to order the start of power supply from the control portion 11 (hereinafter, referred to as enable signal). In the example of FIG. 4, an example in which the control portion 11 supplies the enable signal in order of power supply portions D3, D1, and D2 after the end of the adjustment period is depicted. First, power is supplied to the power supply terminal C3 and for example power from the power supply terminal C3 is supplied to an FPGA for control disposed in the endoscope 20. Subsequently, power is supplied to the endoscope 20 via the power supply terminal C1. Power is supplied to the endoscope 20 via the power supply terminal C2 at last. The control portion 11 temporarily stops all of outputs of the plurality of the power supplies before the switches are turned on by the control portion.

As just described, the switches are disposed in the present embodiment. The switches block the provision of the power supply output from the endoscope power supply to the endoscope. The switches are set to the off-state in an adjustment period. Adjustment is carried out to cause the power supply outputs of the endoscope power supplies to become the target output voltages in the adjustment period. Thereby, the power supply output that causes trouble in electronic components in the endoscope is prevented from being provided to the endoscope. Due to this, even in the case of changing the supply voltage provided to the endoscope, such as the case in which a different kind of endoscope is connected, the necessary power supply outputs can be provided to the endoscope without adversely affecting components of the endoscope.

Furthermore, after the end of the adjustment period, the power supply outputs of the endoscope power supplies are temporarily stopped before the switches are turned on. Thus, stable power supply using the soft start function based on the endoscope power supply is possible.

FIG. 5 is a block diagram depicting a second embodiment of the present invention. In FIG. 5, the same constituent element as FIG. 1 is given the same numeral reference and description thereof is omitted. In FIG. 5, only the power supply portion D of one system is depicted for simplification of the drawing. However, the number of power supply portions D is set to the maximum value of the number of power supplies needed for the connectable endoscope. The present embodiment generates a power supply output set in consideration of the amount of voltage drop due to the switch 13.

In a CCU 21 of the present embodiment, the voltage detecting portion 14 detects not only the voltage of the output terminal of the endoscope power supply 12, or input terminal of the switch 13, but also the voltage of the output terminal of the switch 13 and outputs the detection result to the control portion 11. In other words, the voltage detecting portion 14 detects a voltage across the switch 13. Based on the detection result of the voltage detecting portion 14, the control portion 11 obtains the amount of voltage drop due to the switch 13. The control portion 11 sets the target output voltage of the power supply output of the endoscope power supply 12 higher in consideration of the amount of voltage drop. As a result, the control portion 11 controls the power supply output voltage of the endoscope power supply 12 in such a manner that the voltage at the output terminal of the switch 13 corresponds with the original target output voltage. In other words, the control portion 11 adjusts an output voltage of the power supply 12 in such a manner as to compensate for an amount of voltage drop due to the switch 13 based on the detection result.

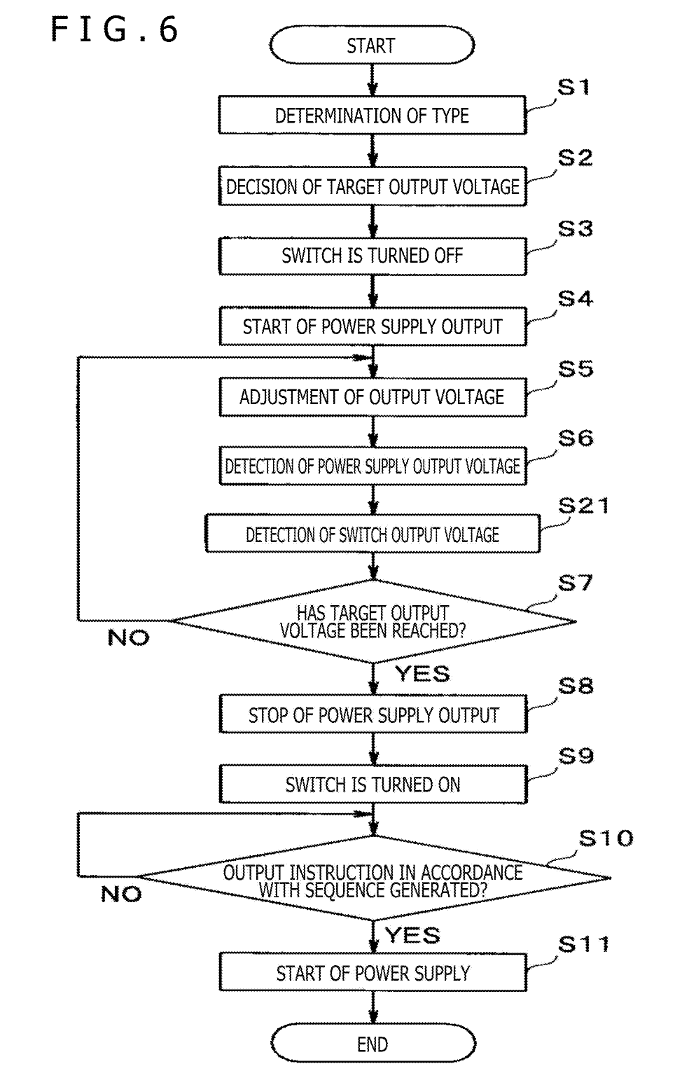

Next, operation of the embodiment configured in this manner will be described with reference to a flowchart of FIG. 6. In FIG. 6, the same step as FIG. 3 is given the same numeral reference and description thereof is omitted.

The processing of Steps S1 to S6 in FIG. 6 is the same as the first embodiment. The voltage detecting portion 14 detects the voltage at the input terminal of the switch 13. Furthermore, the voltage detecting portion 14 detects also the voltage at the output terminal of the switch 13 in Step S21. Based on the detection result of the voltage detecting portion 14, the control portion 11 (i) obtains the amount of voltage drop due to the switch 13 and (ii) sets the target output voltage of the power supply output of the endoscope power supply 12 higher in consideration of the amount of voltage drop. The control portion 11 controls the power supply output voltage of the endoscope power supply 12 in such a manner that the voltage at the output terminal of the switch 13 corresponds with the original target output voltage. If the control portion 11 determines that the voltage at the output terminal of the switch 13 has corresponded with the original target output voltage in Step S7, the control portion 11 makes a transition of the processing to the next Step S8. The other operation is the same as the first embodiment.

As just described, the same effects as the first embodiment can be obtained also in the present embodiment. Furthermore, in the present embodiment, voltage adjustment in consideration of the amount of voltage drop due to the switch that blocks power supply to the endoscope is carried out. Thus, the present embodiment has an effect that the “power supply output” that should be provided to the endoscope can be surely obtained.

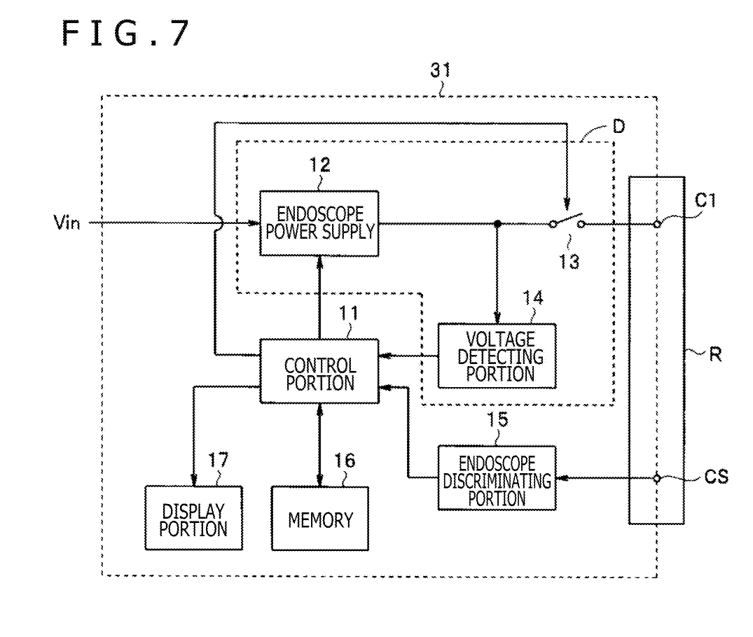

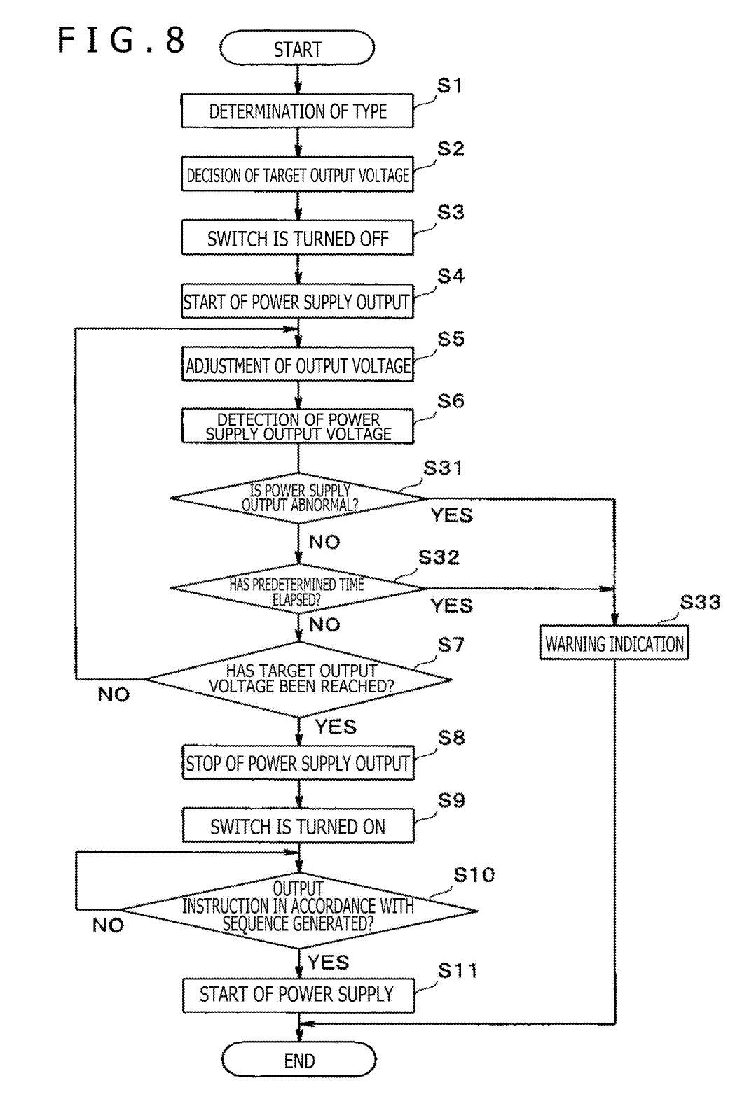

FIG. 7 is a block diagram depicting a third embodiment of the present invention. In FIG. 7, the same constituent element as FIG. 1 is given the same numeral reference and description thereof is omitted. In FIG. 7, only the power supply portion D of one system is depicted for simplification of the drawing. However, the number of power supply portions D is set to the maximum value of the number of power supplies needed for the connectable endoscope. The present embodiment enables presentation of a breakdown of the endoscope power supply 12.

In a CCU 31 of the present embodiment, the endoscope power supply 12 is supplied with a direct-current input voltage Vin to operate and generate a direct-current power supply output. Information on this direct-current input voltage Vin is stored in the memory 16. The control portion 11 determines a breakdown of the endoscope power supply 12 by (i) reading out the information on the direct-current input voltage Vin from the memory 16 and (ii) comparing the direct-current input voltage Vin with the power supply output.

For example, if the “power supply output” with the same voltage as the direct-current input voltage Vin is generated from the output terminal of the endoscope power supply 12, the control portion 11 determines that a conduction breakdown between the input terminal and the output terminal has occurred in the endoscope power supply 12. Furthermore, if the output terminal of the endoscope power supply 12 is at 0 V, the control portion 11 determines that the output terminal of the endoscope power supply 12 is in the open state. Moreover, the control portion may (i) use time information from a timer that is not depicted in the diagram and (ii) determine that a breakdown has occurred in the endoscope power supply 12 if the power supply output with a desired voltage is not obtained even after the elapse of a predetermined time from an order to start generation of the power supply output to the endoscope power supply 12.

If determining that a breakdown has occurred in the endoscope power supply 12, the control portion 11 causes a display portion 17 to display a warning indication depicting whether a breakdown exists and the contents of the breakdown. The display portion 17 is formed of a liquid crystal display (LCD) or the like and is controlled by the control portion 11 to display the warning indication.

Next, operation of the embodiment configured in this manner will be described with reference to a flowchart of FIG. 8.

By powering-on of the CCU 10, the direct-current input voltage Vin is supplied to the endoscope power supply 12. In Step S4 in FIG. 8, the control portion 11 causes the endoscope power supply 12 to start generation of the power supply output. The endoscope power supply 12 adjusts the power supply output so that the target output voltage specified by the control portion 11 may be obtained in Step S5. The control portion 11 causes the output voltage of the endoscope power supply 12 to be detected by the voltage detecting portion 14 in Step S6.

The control portion 11 as breakdown detecting means determines whether or not a breakdown has occurred in the endoscope power supply 12 based on the detection result of the voltage detecting portion 14. For example, the control portion 11 determines an abnormality of the power supply output in Step S31. If the voltage detection result depicting that the power supply output corresponds with the direct-current input voltage Vin is obtained, the control portion 11 (i) determines that a conduction breakdown has occurred in the endoscope power supply 12 and, in Step S33, (ii) displays a “warning indicating” on the display screen of the display portion 17. The “warning indicating” depicts the conduction breakdown has occurred in the endoscope power supply 12. Furthermore, if the power supply output does not increase from a value near 0 V, the control portion 11 determines that the output terminal of the endoscope power supply 12 is in the open state, and the control portion 11 displays a warning indicating depicting that a conduction breakdown of the endoscope power supply 12 has occurred on the display screen of the display portion 17.

Moreover, in Step S32, the control portion 11 determines whether or not a predetermined period has elapsed from the start of generation of the power supply output in Step S4. If the power supply output does not reach the desired target output voltage even after the elapse of this predetermined period, the control portion 11 determines that the endoscope power supply 12 is broken down and displays a warning indication depicting this on the display screen of the display portion 17 in Step S33. The other operation is the same as the first embodiment.

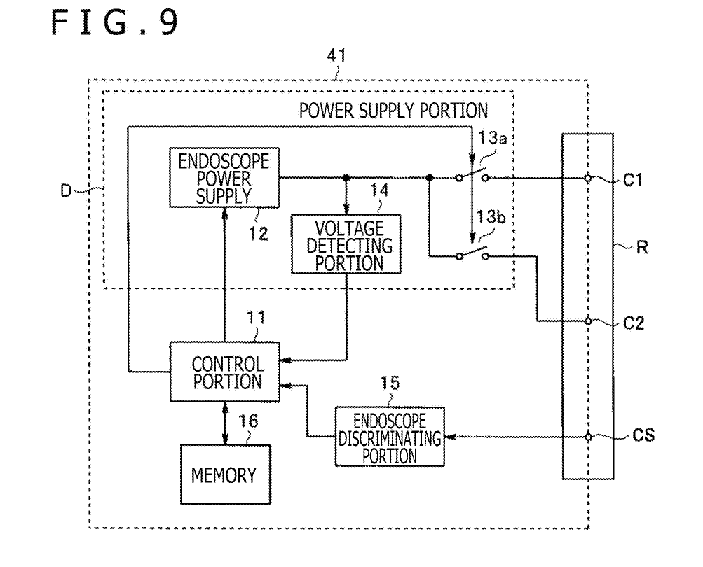

As just described, the same effects as the respective embodiments described hereinbefore can be obtained in the present embodiment. Furthermore, in the present embodiment, the switch 13 is in the off-state in the adjustment period of the power supply output. Thus, an abnormal power supply output is not provided to the endoscope even when an abnormality has occurred in the power supply output due to a breakdown of the endoscope power supply 12 or the like. Moreover, in the present embodiment, there is an advantage that a warning indication regarding such a breakdown can be displayed. FIG. 9 is a block diagram depicting a fourth embodiment of the present invention. In FIG. 9, the same constituent element as FIG. 1 is given the same numeral reference and description thereof is omitted. In FIG. 9, only the power supply portion D of one system is depicted for simplification of the drawing. However, the number of power supply portions D is set to a number that corresponds to the number of power supplies needed for the connectable endoscope and is smaller than the maximum number. The present embodiment is what is configured in such a manner that power can be supplied to multiple power supply lines of an endoscope by the power supply portion D of one system.

A CCU 41 of the present embodiment is a unit that employs multiple switches 13a, 13b, . . . in the power supply portion D. In FIG. 9, an example in which two switches 13a and 13b are employed is depicted. The switches 13a and 13b are disposed on the respective power supply lines between the output terminal of the endoscope power supply 12 and the power supply terminal C1 or C2. The switches 13a and 13b can be on- or off-controlled by the control portion 11. The switches 13a and 13b can permit or block provision of the power supply output from the endoscope power supply 12 to the endoscope 20. The control portion 11 controls the on- and off-states of the switches 13a and 13b based on a type determination result of an endoscope.

In the embodiment configured in this manner, power can be supplied to power supply lines of multiple systems (in FIG. 9, two systems) of an endoscope by the power supply portion D of one system. For example, suppose that endoscope A has a power supply line connected to the power supply terminal C1 and supplies power (supply voltage 3 V) to a predetermined electronic component through this power supply line, whereas a power supply line that receives power supply from the power supply terminal C2 does not exist. Furthermore, suppose that endoscope B has a power supply line connected to the power supply terminal C2 and supplies power (supply voltage 5 V) to a predetermined electronic component through this power supply line, whereas a power supply line that receives power supply from the power supply terminal C1 does not exist.

For example, when endoscope “A” is connected to the CCU 41, the control portion 11 sets 3 V in the endoscope power supply 12 as the target output voltage based on a type determination result by the endoscope identifying portion 15. After the “power supply output” from the endoscope power supply 12 has reached 3 V, the control portion 11 turns on the switch 13a. The switch 13b remains in the off-state. Due to this, the “power supply output” of 3 V from the endoscope power supply 12 is provided to the predetermined electronic component via the power supply line in endoscope “A” connected to the power supply terminal C1.

Furthermore, when endoscope “B” is connected to the CCU 41, the control portion 11 sets 5 V in the endoscope power supply 12 as the target output voltage based on a type determination result by the endoscope identifying portion 15. After the power supply output from the endoscope power supply 12 has reached 5 V, the control portion 11 turns on the switch 13b. The switch 13a remains in the off-state. Due to this, the power supply output of 5 V from the endoscope power supply 12 is provided to the predetermined electronic component via the power supply line in endoscope “B” connected to the power supply terminal C2. The other operation is the same as the first embodiment.

As just described, the same effects as the first embodiment can be obtained also in the present embodiment. Furthermore, in the present embodiment, the necessary power can be supplied by the power supply portions the number of which is smaller than the maximum number of power supplies necessary for the connectable endoscope.

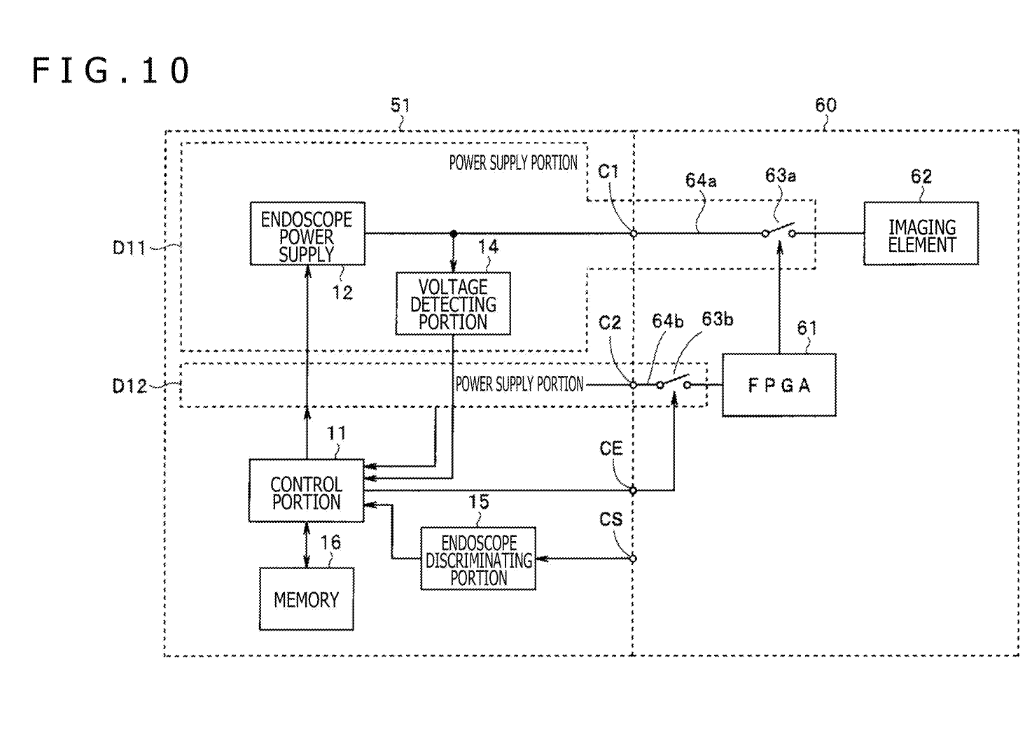

FIG. 10 is a block diagram depicting a fifth embodiment of the present invention. In FIG. 10, the same constituent element as FIG. 1 is given the same numeral reference and description thereof is omitted. Although FIG. 10 depicts an example in which a CCU 51 has power supply portions D11 and D12 of two systems, the number of power supply portions D is set to a number corresponding to the number (maximum number) of power supplies needed for the connectable endoscope. The present embodiment is an example in which switches that supply or block power from the power supply portions D to the respective electronic components of an endoscope are disposed in the endoscope.

In FIG. 10, an endoscope 60 and the CCU 51 are electrically connected. The power supply terminals C1 and C2 of the CCU 51 are connected to power supply lines 64a and 64b, respectively, disposed in the endoscope 60. An imaging element 62 is connected to the power supply line 64a and an FPGA 61 that controls the endoscope 60 is connected to the power supply line 64b.

The power supply portions D11 and D12 have the same configuration as each other and each have the endoscope power supply 12 and the voltage detecting portion 14 in the CCU 51. Furthermore, the power supply portion D11 has a switch 63a on the power supply line 64a of the endoscope 60 and the power supply portion D12 has a switch 63b on the power supply line 64b of the endoscope 60. If the switches 63a and 63b do not need to be discriminated, these switches will be referred to as the switches 63.

A power supply output generated by the endoscope power supply 12 of the power supply portion D11 is provided from the power supply terminal C1 to the imaging element 62 via the power supply line 64a in the endoscope 60 and the switch 63a disposed on the power supply line 64a. Similarly, a “power supply output” generated by the endoscope power supply 12 of the power supply portion D12 is provided from the power supply terminal C2 to the FPGA 61 via (i) the power supply line 64b in the endoscope 60 and (ii) the switch 63b disposed on the power supply line 64b. The switch 63b is on- or off-controlled by a control signal from the control portion 11 input via a control terminal CE of the CCU 51. Furthermore, the FPGA 61 generates a control signal for on- or off-controlling the switch 63a.

Also in the embodiment configured in this manner, the switches 63 are in the off-state in the adjustment period of the endoscope power supplies 12 and are turned on after the end of the adjustment period. In the present embodiment, the switch 63b is controlled by the control portion 11, whereas the switch 63a is controlled by the FPGA 61 in the endoscope 60. After the end of the adjustment period, the control portion 11 (i) generates the control signal to turn on the switch 63b and (ii) supplies the control signal to the switch 63b in the endoscope 60 through the control terminal CE. Thereby, the switch 63b is turned on.

After the switch 63b is turned on, the control portion 11 (i) supplies an enable signal to the endoscope power supply 12 of the power supply portion D12 and (ii) causes the endoscope power supply 12 to generate the power supply output for the FPGA 61. This “power supply output” is provided to the FPGA 61 via the power supply terminal C2, the power supply line 64b, and the switch 63b. Thereby, the FPGA 61 starts operation. After the operation start, the FPGA 61 generates the control signal for turning on the switch 63a. The switch 63a is turned on by this control signal.

The control portion 11 (i) supplies an enable signal to the endoscope power supply 12 of the power supply portion D11 and (ii) causes the endoscope power supply 12 to generate the power supply output for the imaging element 62. This power supply output is provided to the imaging element 62 via the power supply terminal C1, the power supply line 64a, and the switch 63a. Thereby, the imaging element 62 starts operation.

The example in which the CCU has the power supply portions of two systems is depicted in FIG. 10. However, if the CCU has the power supply portions with the number of systems equal to or larger than three systems, the switches other than the switch that controls provision or blocking of power supply to the FPGA may be configured to be on- or off-controlled by the FPGA. Furthermore, if there is room for the number of connection terminals, all switches disposed in the endoscope may be allowed to be controlled by the control portion in the CCU.

As just described, the same effects as the first embodiment are obtained in the present embodiment. Furthermore, in the present embodiment, the switches that control provision or blocking of power supply can be disposed in the endoscope.

In sum, a camera control unit for a plurality of various endoscopes each of which having different voltage requirement and are connectable to the camera control unit is disclosed. The camera control unit comprises a power supply that generates a required voltage for each of the plurality of the various endoscopes expected to be connected to the camera control unit. An identifying portion that identifies the required voltage for one of the plurality of the various endoscopes is connected to the camera control unit and outputs an identification result. A control portion that controls the power supply in such a manner that power having the required voltage is generated according to the identification result. A switch that disposed on a power supply path is connected to the power supply for adjustment when control of the power supply is carried out and is configured to alternate between conduction and blocking of the power supply path. A voltage detecting portion that detects a voltage across the switch and output a detection result. The control portion adjusts an output voltage of the power supply in such a manner as to compensate for an amount of voltage drop due to the switch based on the detection result.

The control portion controls such that to set the switch to an off-state and block the power supply path in an adjustment period for the power supply so as to generate power having the required voltage according to the identification result and turn on the switch and establish conduction of the power supply path after the adjustment period ends. The control portion detects a breakdown of the power supply in the adjustment period.

The camera control unit for an endoscope further comprises a memory that stores an adjustment value for causing the power supply to generate the power having the required voltage according to the identification result in the adjustment period. The control portion controls turning of the switch after the adjustment value is stored in the memory. The control portion generates the power having the required voltage according to the identification result based on the adjustment value stored in the memory after the switch is turned on. The endoscope has a plurality of circuit components operated by a predetermined required voltage. The number of the power supplies, the number of the power supply paths, and the number of the switches correspond to the numbers of the circuit components. The control portion causes a plurality of the power supplies to sequentially generate power having the required voltage in accordance with a predetermined sequence. The control portion temporarily stops all of outputs of the plurality of the power supplies before the switches are turned on by the control portion.

An endoscope system comprises an endoscope and a camera control unit used therein. A plurality of various endoscopes each of which having different voltage requirement are being connectable to the camera control unit. The camera control unit includes a power supply that generates a required voltage for each of the plurality of the various endoscopes expected to be connected to the camera control unit. An identifying portion that identifies the required voltage for one of the plurality of the various endoscopes is connected to the camera control unit and outputs an identification result. A control portion that controls the power supply in such a manner that power having the required voltage of the connected endoscope is generated according to the identification result. A switch disposed on a power supply path is connected to the power supply for adjustment when control of the power supply is carried out and is configured to alternate between switch conduction and blocking of the power supply path. A voltage detecting portion detects a voltage across the switch and output a detection result. The control portion adjusts an output voltage of the power supply in such a manner as to compensate for an amount of voltage drop due to the switch based on the detection result.

The technology disclosed herein is not directly limited to the respective embodiments described hereinbefore and can be embodied with a constituent element modified within such a range as not to depart from the gist thereof at the stage of implementation. Furthermore, various inventions can be formed based on appropriate combinations of multiple constituent elements disclosed in the respective embodiments described hereinbefore. For example, several constituent elements of all constituent elements depicted in the embodiment may be deleted. Moreover, constituent elements that range in different embodiments may be combined as appropriate.

The present disclosure has an effect that components of an endoscope can be prevented from being adversely affected even when outputs of power supplies are adjusted and multiple output voltages are supplied to the endoscope.

What is claimed is:

1. A camera control unit for an endoscope, a plurality of various endoscopes each of which having different voltage requirement being connectable to the camera control unit, the camera control unit comprising:
a power supply that generates a required voltage for each of the plurality of the various endoscopes expected to be connected to the camera control unit;
an identifying portion that identifies the required voltage for one of the plurality of the various endoscopes connected to the camera control unit and outputs an identification result;
a control portion that controls the power supply in such a manner that power having the required voltage is generated according to the identification result;
a switch that is disposed on a power supply path connected to the power supply for adjustment when control of the power supply is carried out, and is configured to alternate between conduction and blocking of the power supply path; and
a voltage detecting portion that detects a voltage across the switch and output a detection result,
wherein the control portion adjusts an output voltage of the power supply in such a manner as to compensate for an amount of voltage drop due to the switch based on the detection result.

2. The camera control unit for an endoscope of claim 1, wherein the control portion controls such that to:
set the switch to an off-state and block the power supply path in an adjustment period for the power supply so as to generate power having the required voltage according to the identification result, and
turn on the switch and establish conduction of the power supply path after the adjustment period ends.

3. The camera control unit for an endoscope of claim 2, wherein the control portion detects a breakdown of the power supply in the adjustment period.

4. The camera control unit for an endoscope of claim 2 further comprising:
a memory that stores an adjustment value for causing the power supply to generate the power having the required voltage according to the identification result in the adjustment period and
wherein the control portion controls turning of the switch after the adjustment value is stored in the memory, and
the control portion generates the power having the required voltage according to the identification result based on the adjustment value stored in the memory after the switch is turned on.

5. The camera control unit for an endoscope of claim 2,
wherein the endoscope has a plurality of circuit components operated by a predetermined required voltage,
the number of the power supplies, the number of the power supply paths, and the number of the switches correspond to the numbers of the circuit components, and
the control portion causes a plurality of the power supplies to sequentially generate power having the required voltage in accordance with a predetermined sequence.

6. The camera control unit for an endoscope of claim 5,
wherein the control portion temporarily stops all of outputs of the plurality of the power supplies before the switches are turned on by the control portion.

7. An endoscope system comprising:
an endoscope; and
a camera control unit for the endoscope, a plurality of various endoscopes each of which having different voltage requirement being connectable to the camera control unit, the camera control unit includes:
a power supply that generates a required voltage for each of the plurality of the various endoscopes expected to be connected to the camera control unit;
an identifying portion that identifies the required voltage for one of the plurality of the various endoscopes connected to the camera control unit and outputs an identification result;
a control portion that controls the power supply in such a manner that power having the required voltage of the connected endoscope is generated according to the identification result;
a switch that is disposed on a power supply path connected to the power supply for adjustment when control of the power supply is carried out, and is configured to alternate between switch conduction and blocking of the power supply path; and
a voltage detecting portion that detects a voltage across the switch and output a detection result,
wherein the control portion adjusts an output voltage of the power supply in such a manner as to
compensate for an amount of voltage drop due to the switch based on the detection result.
